(19) United States
(12) Patent Application Publication
(10) Pub. No.: US 2026/0224414 A1
(43) Pub. Date: Aug. 6, 2026

(54) Insertion Aid Apparatus for Replaceable Inserts and Padding

(71) Applicant: Rocio Isabel Laca, Las Vegas, NV (US)

(72) Inventor: Rocio Isabel Laca, Las Vegas, NV (US)

(21) Appl. No.: 19/575,288

(22) Filed: Mar. 23, 2026

Related U.S. Application Data
(63) Continuation-in-part of application No. 19/192,193, filed on Apr. 28, 2025.
(60) Provisional application No. 63/658,847, filed on Jun. 11, 2024.

(51) Int. Cl. A61F 13/49

(52) U.S. Cl. CPC A61F 13/49058; A61F 13/49006

(57) ABSTRACT

A diaper insert insertion aid apparatus is disclosed, designed to streamline and simplify the process of inserting absorbent inserts into cloth diaper covers. The apparatus comprises a main body configured to hold multiple reusable cloth diaper inserts, a fastening mechanism to secure the inserts in place, and resilient pads for stability. In one embodiment, the main body further includes a handle for ease of manipulation. The apparatus can accommodate inserts with or without pockets, and the pocketed inserts can be further supported by a dedicated structure within the apparatus. This invention aims to address the inconvenience and physical strain associated with traditional manual insertion methods, making cloth diapering more accessible and appealing to a wider range of users, including those with limited dexterity or handling multiple diapers. The apparatus is adaptable to various insert configurations and can be used to place replaceable inserts in other fabric pocket containing devices.

### BACKGROUND OF THE INVENTION

The present invention relates to the field of cloth diapering, specifically to devices and methods for facilitating the insertion of absorbent inserts into cloth diaper covers.

Cloth diapering has gained popularity in recent years due to its environmental and economic benefits compared to disposable diapers. A common type of cloth diaper system involves a reusable outer cover or “shell” and absorbent inserts that are placed inside the cover. These inserts can be made of various materials such as cotton or microfiber, and come in different shapes and sizes.

A prevalent design for cloth diaper inserts is the rectangular shape. While this shape fits well within most diaper covers, it presents challenges during the insertion process. Users must manually stuff the inserts into the diaper cover, often requiring significant dexterity and hand strength. This can be particularly difficult for individuals with large hands, limited hand mobility, or conditions such as carpal tunnel syndrome.

Furthermore, many families using cloth diapers own dozens of diapers to accommodate frequent changes. The cumulative time and effort required to manually stuff these numerous inserts can become a significant burden, discouraging some parents from using cloth diapers altogether.

Several attempts have been made to address this issue. Some manufacturers offer pre-stuffed diaper covers, where the absorbent layers are sewn directly into the cover. However, this limits the flexibility of adjusting absorbency levels and can make the covers bulky and difficult to clean.

Other solutions include using diaper fasteners or pins to secure the inserts in place, but these can be cumbersome and potentially irritate the baby's skin. Additionally, some inserts are designed with snap closures or tabs, but these add complexity to the manufacturing process and can increase the cost of the product.

The current methods for inserting cloth diaper inserts are time-consuming, physically demanding, and inconvenient for many users. Therefore, is a clear need for a more efficient and user-friendly solution that simplifies the insertion process, reduces physical strain, and promotes the wider adoption of cloth diapers.

### SUMMARY OF THE INVENTION

The present invention addresses these shortcomings by providing a diaper insert insertion aid apparatus and a modified cloth diaper insert design that work together to streamline and simplify the insertion process. The present invention aims to make cloth diapering more accessible and appealing to a wider range of users by reducing the manual effort and time required for diaper preparation.

The diaper insert insertion aid apparatus of the present invention comprises a main body configured to receive and hold a plurality of reusable cloth diaper inserts, wherein the main body is configured for insertion into a diaper cover. It further comprises a fastening mechanism integrated into the main body, configured to securely hold the plurality of diaper inserts in place within the main body during insertion, said fastening mechanism comprising at least one hook-and-loop fastener. Also, a plurality of resilient pads is attached to an outer surface of the main body, configured to provide stability and frictional contact with a surface upon which the apparatus is placed.

According to an embodiment of the present invention, the main body is configured to accommodate up to three reusable cloth diaper inserts.

According to another embodiment of the present invention, the diaper insert insertion aid apparatus for a diaper insert with pocket comprises a main body configured to receive and hold at least one reusable cloth diaper insert, a pocket formed within the said reusable cloth diaper insert, the pocket having an opening and being located on a first side of the insert to facilitate gripping and insertion of the insert into a diaper cover and a plurality of resilient pads attached to the main body, configured to provide stability and frictional contact with a surface upon which the apparatus is placed.

The present invention also discloses a reusable cloth diaper insert for use with a diaper insert insertion aid apparatus. The insert comprises a rectangular body formed from a plurality of layers of absorbent fabric material and a pocket formed on a first side of the rectangular body, the pocket having an opening configured to facilitate manual insertion of the insert into a diaper cover.

According to an embodiment of the present invention, the pocket is formed by folding a portion of the rectangular body and securing it with a stitching pattern.

In another embodiment of the present invention, the insertion aid apparatus may be utilized to place a replaceable insert into a corresponding fabric pocket of various items including sports bras, athletic wear, swimwear, reusable feminine products, medical garments, protective sports apparel, adult incontinence garments/underwear, duvet covers, and pillow covers. The insertion aid apparatus may be utilized to place a replaceable insert in a corresponding fabric or deformable pocket of any other item with such a configuration.

### BRIEF DESCRIPTION OF THE DRAWINGS

FIG. 1 is a perspective view of a first embodiment of the insertion aid apparatus with diaper inserts installed.

FIG. 2 is a perspective view of a first embodiment of the insertion aid apparatus without diaper inserts installed.

FIG. 3 is a bottom perspective view of a first embodiment of the insertion aid apparatus without diaper inserts installed.

FIG. 4 is a perspective view of a first embodiment of the insertion aid apparatus with diaper inserts installed.

FIG. 5 is a front view of a first embodiment of the insertion aid apparatus without diaper inserts installed.

FIG. 6 is a rear view of a first embodiment of the insertion aid apparatus without diaper inserts installed.

FIG. 7 is a side view of a first embodiment of the insertion aid apparatus without diaper inserts installed.

FIG. 8 is a top view of a first embodiment of the insertion aid apparatus without diaper inserts installed.

FIG. 9 is a bottom view of a first embodiment of the insertion aid apparatus without diaper inserts installed.

FIG. 10 is a perspective view of a second embodiment of the insertion aid apparatus with diaper inserts installed.

FIG. 11 is a bottom perspective view of a second embodiment of the insertion aid apparatus with diaper inserts installed.

FIG. 12 is a top view of a second embodiment of the insertion aid apparatus without diaper inserts installed.

FIG. 13 is a rear view of a second embodiment of the insertion aid apparatus with a diaper insert installed.

FIG. 14 is a side view of a second embodiment of the insertion aid apparatus with a diaper insert installed.

### DESCRIPTION OF THE PREFERRED EMBODIMENT

The following detailed description refers to the preferred embodiment of the disclosed invention as shown in the attached figures and in the below description. This detailed description is not meant to limit the scope of the invention in any way but is intended to disclose the preferred embodiment/best mode of the invention at the time of filing this application.

In the following description of the embodiments of the invention, reference is made to the accompanying drawings that form a part hereof, and which are shown by way of illustration of specific embodiments in which the invention may be practiced. These embodiments are described in sufficient detail to enable those skilled in the art to practice the invention, and it is to be understood that other embodiments may be utilized and that changes may be made without departing from the scope of the present invention.

The specification may refer to “an”, “one” or “some” embodiment(s) in several locations. This does not necessarily imply that each such reference is to the same embodiment(s), or that the feature only applies to a single embodiment. Single feature of different embodiments may also be combined to provide other embodiments.

As used herein, the singular forms “a”, “an” and “the” are intended to include the plural forms as well unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries should be interpreted as having a meaning that is consistent with their meaning in the context of the relevant art and will not be interpreted in an idealized or overly formal sense unless expressly so defined herein.

The utility of the system described herein will be explained further in detail in the following sections of this document referring to the figures. Specific terms used herein do not restrict the scope of the present disclosure.

Embodiments of the present invention disclose a diaper insert insertion aid apparatus.

The present invention addresses the difficulties associated with the manual insertion of absorbent diaper inserts into cloth diaper covers. Current methods are often time-consuming, laborious, and inconvenient, especially for those with limited dexterity or when dealing with multiple diapers. This invention seeks to streamline and simplify the insertion process, making cloth diapering more accessible and appealing to a wider range of users.

The main feature of the present invention lies in an insertion aid apparatus designed to receive and hold multiple reusable cloth diaper inserts. This apparatus features a main body configured for insertion into the diaper cover. To prevent slippage and maintain stability during use, the outer surface of the main body is equipped with a plurality of slip resistant pads, preferably made of silicone, rubber, or elastomer. Additionally, a fastening mechanism, such as a hook-and-loop fastener (e.g., Velcro), is integrated into the main body to securely hold the inserts in place during the insertion process.

In an embodiment, the apparatus further optionally incorporates a handle portion extending from the bottom section of the main body. This handle provides enhanced leverage and control, facilitating easier manual manipulation and precise guidance of the diaper inserts into the cover.

Alternatively, the invention provides an embodiment where the diaper insert itself is modified to include a pocket on one side. This pocket functions as a convenient grip point for the user and further simplifies the insertion process. The apparatus can be configured to accommodate this modified insert seamlessly.

The apparatus is configured to hold multiple inserts, typically up to three, enabling efficient preparation of several diapers simultaneously.

In embodiments utilizing the pocketed insert, the main body of the apparatus may include a support structure to help maintain the shape and prevent deformation of the insert during insertion, ensuring a smooth and effortless process.

The invention also provides various materials and configurations for the resilient pads and fastening mechanisms. The pads can be made from different types of silicone, rubber, or elastomers, while the fastening mechanism can utilize Velcro, snaps, buttons, or magnets, offering flexibility in design and functionality.

The present invention also includes a reusable cloth diaper insert specifically designed for use with the insertion aid apparatus. The insert comprises a rectangular body made of absorbent fabric material and includes a pocket on one side.

In a preferred embodiment, the pocket is formed by folding a portion of the rectangular body and securing it with stitching. This construction creates a simple, durable, and functional pocket for easy handling during insertion.

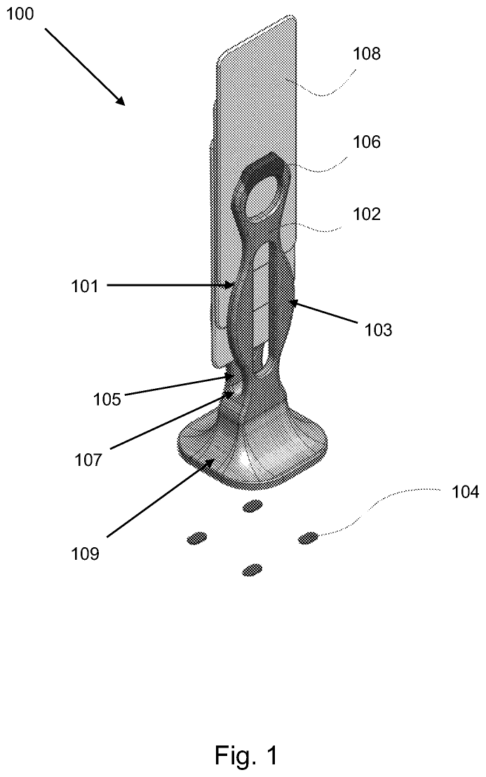

FIG. 1 illustrates a perspective view of an insertion aid apparatus 100 according to the present disclosure. The apparatus 100 includes a main body 102 configured for supporting one or more diaper inserts 108 to ease their insertion into complementary slot in a diaper cover. Main body 102 is comprised of diaper insert slot 101 which is configured to hold or support one or more diaper inserts 108. Diaper insert slot 101 is formed by two vertical support members, first vertical support 103 and second vertical support 105. Insert slot base 107 forms the base of diaper insert slot 101. Insert slot base 107 provides vertical support for diaper insert(s) 108 while located in diaper insert slot 101. First vertical support member 103 and second vertical support member 105 provide lateral support for diaper insert(s) 108 while located in diaper insert slot 101.

Main body 102 and diaper insert slot 101 may be sized/configured to hold one or more diaper inserts 108. In the preferred embodiment, diaper insert slot 101 holds up to three (3) diaper inserts 108. While diaper insert(s) 108 are installed in diaper insert slot 101, a user may utilize the apparatus to ease the insertion of the diaper insert(s) 108 into a complementary slot in a diaper cover. The insertion action is performed by placing the complementary slot in the diaper cover over the apparatus 100, specifically over diaper insert slot 101. Once the diaper cover slot is placed over the diaper insert 108, the diaper insert 108 is secured within the diaper cover slot and is this installed in the diaper cover.

Base 109 of main body 102 provides support to apparatus 100 when apparatus 100 is placed on a surface such as a table or other work service. The stability and lack of slip on a hard smooth surface, of apparatus 100 is important to the best operation of the device when inserting diaper insert(s) 108 into diaper covers. To provide stability, a plurality of slip-resistant pads 104 are attached to the underside of the base 109. Slip-resistant pads 104 provide cushioning and slip resistance for the base 109 of the apparatus 100 during use to insert diaper inserts 108 into diaper covers. In the preferred embodiment, slip resistant pads 104 are formed of silicone, but any material well known in the art and suitable for the purpose could be used such as rubber, PVC, various plastics, elastomer, and/or felt.

The apparatus 100 further comprises a fastening mechanism 106 that integrated into the main body 102. Fastening mechanism 106 is configured to securely hold a plurality of diaper inserts 108 in place within the diaper insert slot 101 during insertion. In the preferred embodiment, fastening mechanism 106 is located at the top of one or both first vertical support 103 and second vertical support 105 and is comprised of a hook and loop fastener. The hook portion is affixed to the main body 102, specifically one or both first vertical support 103 and second vertical support 105. This hook material provides frictional resistance to assist keeping the additional diaper inserts 108 in the diaper insert slot 101 while one is installed in a diaper cover. The hook material of fastening mechanism 106 is attached to main body 102 using any adhesive well known in the art such as various glues or epoxy materials. Any attachment method well known in the art may be utilized. Fastening mechanism 106 may be comprised of various other fasteners well known in the art such as clips, snaps, buttons, raised teeth, frictional texture, or magnets.

Apparatus 100 may be formed of any material well known in the art such as various plastics, woods, metals or any other rigid, stable material well known in the art. In the preferred embodiment, apparatus 100 is formed in molded plastic. Any manufacturing method well known in the art may be used such as molding, cutting, 3d printing, and/or casting. In the preferred embodiment, apparatus 101 is made of molded plastic.

Diaper inserts 108 may be made of any suitable absorbent material well known in the art including cotton, organic cotton, bamboo, hemp, microfiber, microfleece, and non-woven interlining material such as products sold under the Zorb® trademark. Diaper covers may also be made of any suitable material well known in the art including polyester knits, rayon, cotton, organic cotton, bamboo, hemp, microfiber, and microfleece.

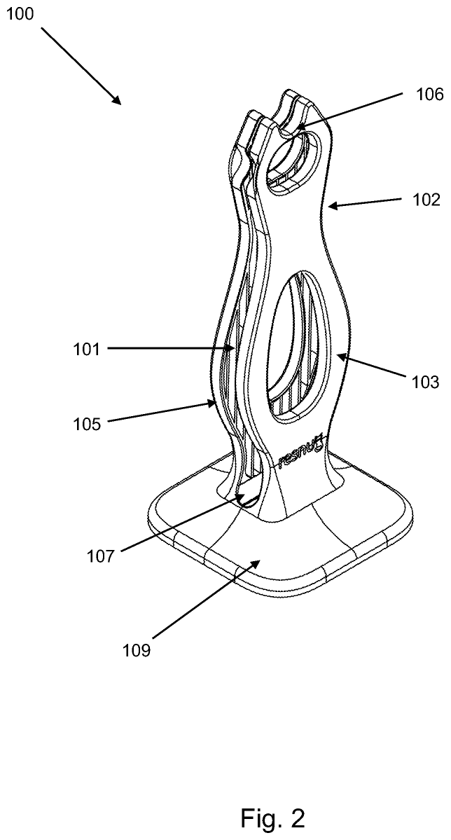

FIG. 2 shows a perspective view of the insertion aid apparatus 100 without diaper inserts 108 installed. In this view, the constituent part of apparatus 100 are shown. Diaper insert slot 101 is formed of first vertical support 103, second vertical support 105 and insert slot base 107. Body 102 of apparatus 100 is supported by base 109. Fastening mechanism 106 is attached at the top of first vertical support 103 and/or second vertical support 105.

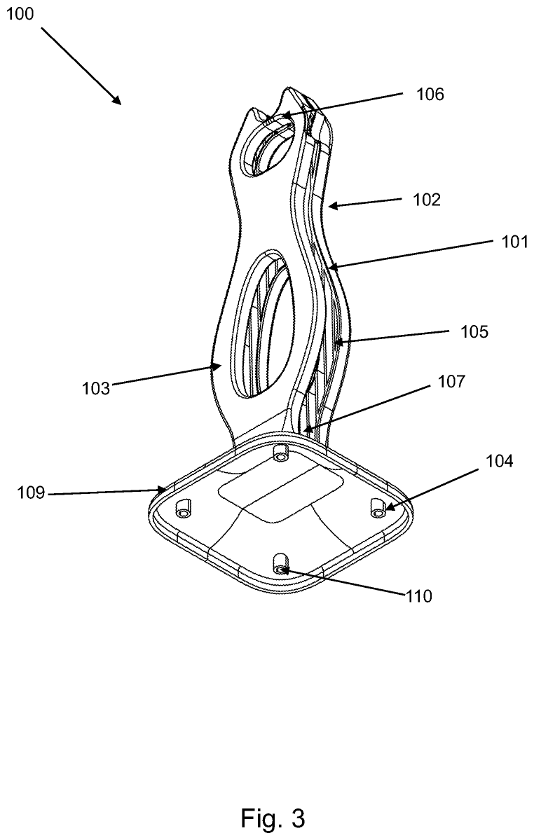

FIG. 3 shows a bottom perspective view of the insertion aid apparatus 100. In this view, the location of slip-resistant pads 104 is shown on the underside of base 109. Slip-resistant pads 104 are positioned to provide stability and frictional contact with a surface on which the apparatus 100 is placed.

Also shown in this view is pad support 110, which provides the installation location for slip-resistant pads 104. Pads 104 may be sized to couple with a complementary opening of pad support 110, thus providing secure installation of pads 104 to base 109. In other embodiments, adhesive may be utilized to secure pads 104 to pad support 110 or base 109. While this configuration of base 109, pad support 110 and pads 104 is disclosed in this embodiment, any configuration or number of pads 104 could be utilized. Any installation or securing method well known in the art may also be utilized.

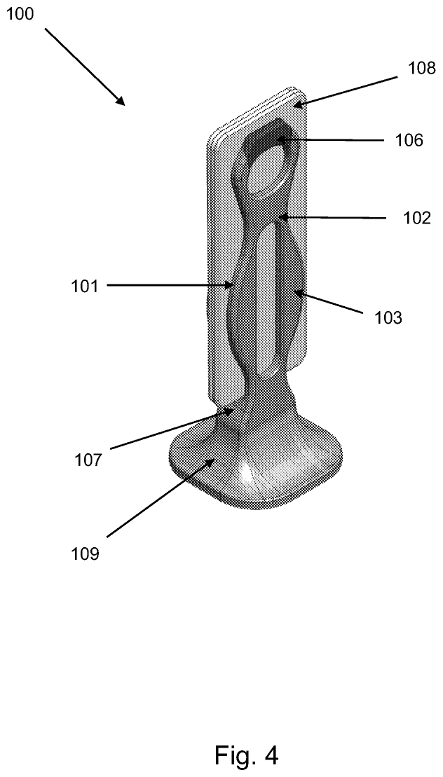

FIG. 4 illustrates another perspective view of the diaper insert insertion aid apparatus 100, highlighting the arrangement of the diaper inserts 108 within the main body 102, diaper insert slot 101 and vertical supports. The engagement of the fastening mechanism 106 is also shown.

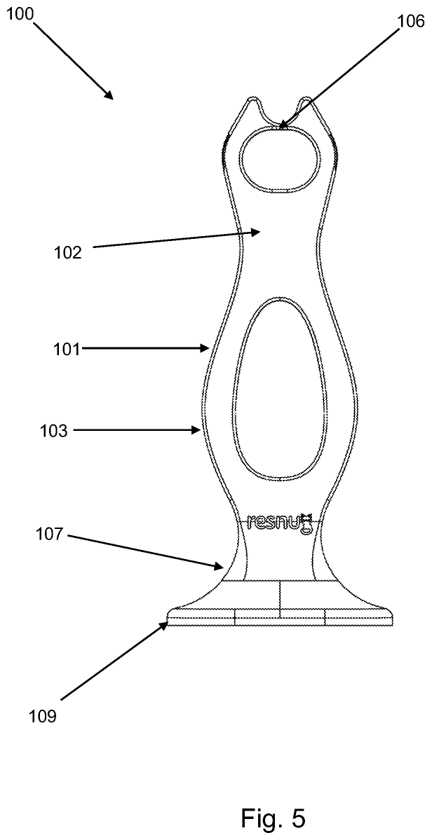

FIG. 5 shows a front view of the insertion aid apparatus 100 with its constituent parts visible. In this view, body 102, diaper insert slot 101, insert slot base 107 and base 109 are shown. Further, fastening mechanism 106 is visible at the top of first vertical support 103. The operation and construction of insert aid apparatus 100 is the same as described in relation to embodiments discussed above.

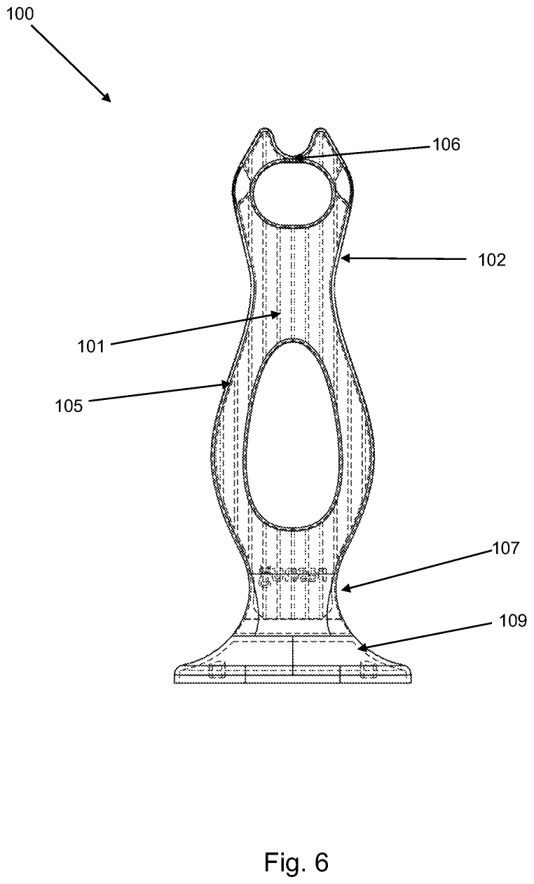

FIG. 6 shows a rear view of the insertion aid apparatus 100 with its constituent parts visible. In this view, body 102, diaper insert slot 101, insert slot base 107 and base 109 are shown. Further, fastening mechanism 106 is visible at the top of second vertical support 105. The operation and construction of insert aid apparatus 100 is the same as described in relation to embodiments discussed above.

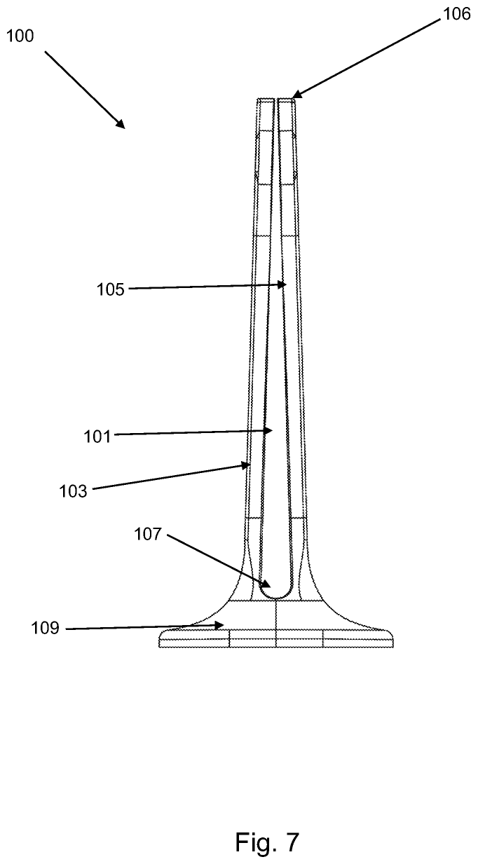

FIG. 7 shows a side view of the insertion aid apparatus 100 with its constituent parts visible. In this view, body 102, diaper insert slot 101, first vertical support 103, second vertical support 105, insert slot base 107 and base 109 are shown. Further, fastening mechanism 106 is visible at the top of first vertical support 103 and second vertical support 105. In this view, diaper insert slot 101 is clearly visible, specifically the location for inserting diaper insert(s) 108 (not shown). This view shows the defined cavity for diaper insert(s) 108 with support from the bottom given by insert slot base 107 and lateral support at the sides provided by first vertical support 103 and second vertical support 105. The operation and construction of insert aid apparatus 100 is the same as described in relation to embodiments discussed above.

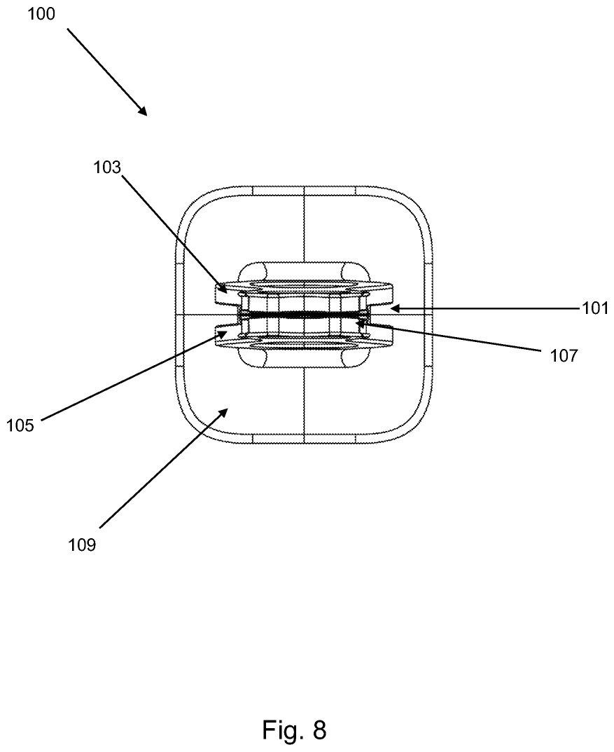

FIG. 8 shows a top view of the insertion aid apparatus 100. In this view, first vertical support 103, second vertical support 105, diaper insert slot 101 and insert slot base 107 are visible. As described above, these elements provide support for diaper inserts 108 (not shown) when inserted in the insertion aid apparatus 100. Base 109 is also shown, providing stable support for apparatus 100.

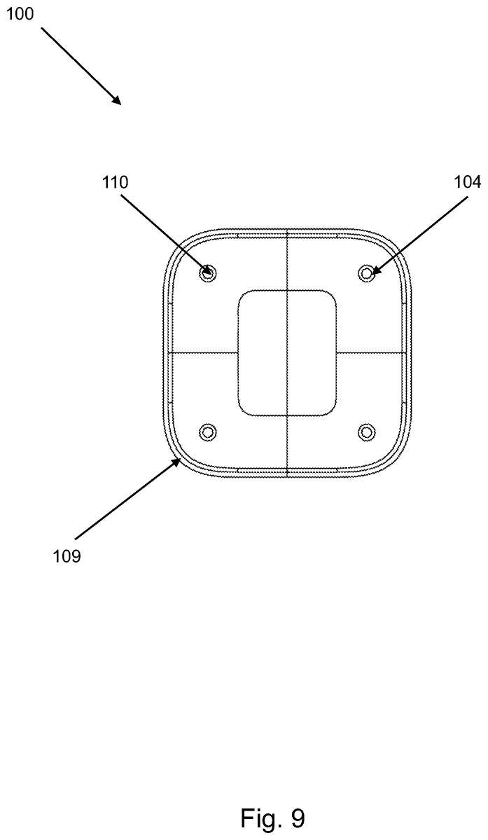

FIG. 9 shows a bottom view of insertion aid apparatus 100. In this view, slip-resistant pads 104 are shown installed in pad support 110 on the underside of base 109. In this configuration, there are four pads 104 and four pad supports 110. This configuration provides stable and frictional/gripping support for the base 109 of apparatus 100.

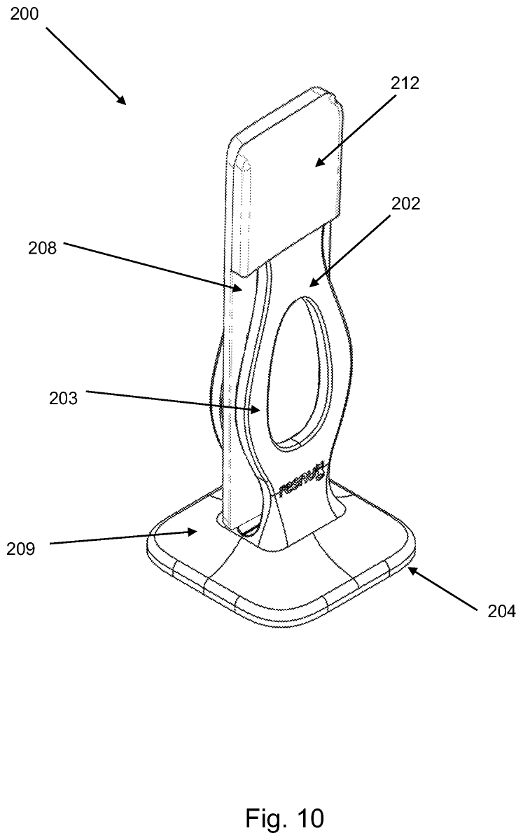

FIG. 10 shows a secondary embodiment of insertion aid apparatus 200. In this embodiment, apparatus 200 is comprised of body 202, base 209 and slip-resistant pads 204. Body 202 is comprised of a single vertical support 203 with an upper portion configured to fit within pocket 212 of diaper insert 208. This embodiment of insertion aid apparatus 200 utilizes a diaper insert 208 with a pocket 212. The user fits the pocket 212 over the top of body 202 and inserts the diaper insert 208 into a diaper cover. The diaper cover can be removed from the apparatus after the diaper insert 208 is secured within the complementary pocket on the diaper cover.

Base 209 of main body 202 provides support and frictional grip to apparatus 200 when apparatus 200 is placed on a surface such as a table or other work service. The stability and lack of slip on a hard smooth surface of apparatus 200 is important to the best operation of the device when inserting diaper insert(s) 208 into diaper covers. To provide stability, a plurality of slip-resistant pads 204 are attached to the underside of the base 209. Slip-resistant pads 204 provide cushioning and slip resistance for the base 209 of the apparatus 200 during use to insert diaper inserts 208 into diaper covers. In the preferred embodiment, slip resistant pads 204 are formed of silicone, but any material well known in the art and suitable for the purpose could be used such as rubber, PVC, various plastics, and/or felt.

Apparatus 200 may be formed of any material well known in the art such as various plastics, woods, metals or any other rigid, stable material well known in the art. In the preferred embodiment, apparatus 200 is formed in molded plastic. Any manufacturing method well known in the art may be used such as molding, cutting, 3d printing, and/or casting. In the preferred embodiment, apparatus 200 is made of molded plastic.

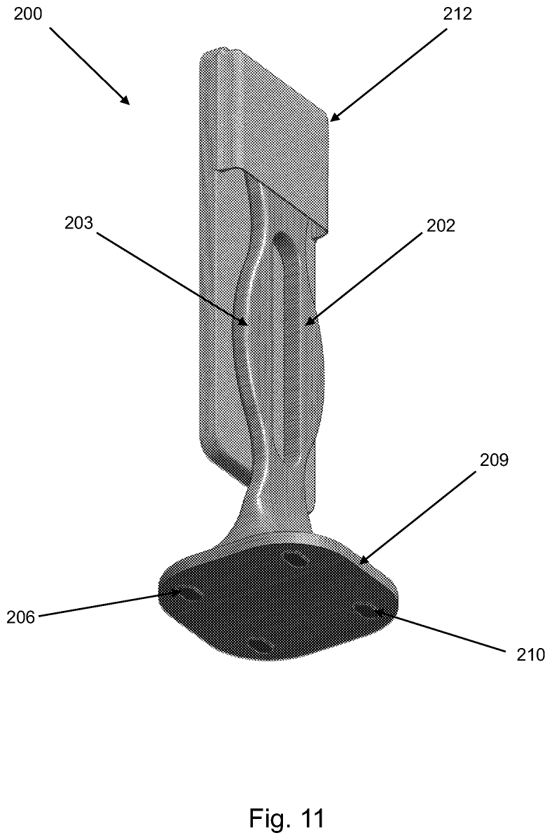

FIG. 11 shows a bottom perspective view of the second embodiment of the insertion aid apparatus 200. In this view, the location of slip-resistant pads 204 is shown on the underside of base 209. Slip-resistant pads 204 are positioned to provide stability and frictional contact with a surface on which the apparatus 200 is placed.

Also shown in this view is pad support 210, which provides the installation location for slip-resistant pads 204. Pads 204 may be sized to couple with a complementary opening of pad support 210, thus providing secure installation of pads 204 to base 209. In other embodiments, adhesive may be utilized to secure pads 204 to pad support 210 or base 209. While this configuration of base 209, pad support 210 and pads 204 is disclosed in this embodiment, any configuration or number of pads 204 could be utilized. Any installation or securing method well known in the art may also be utilized.

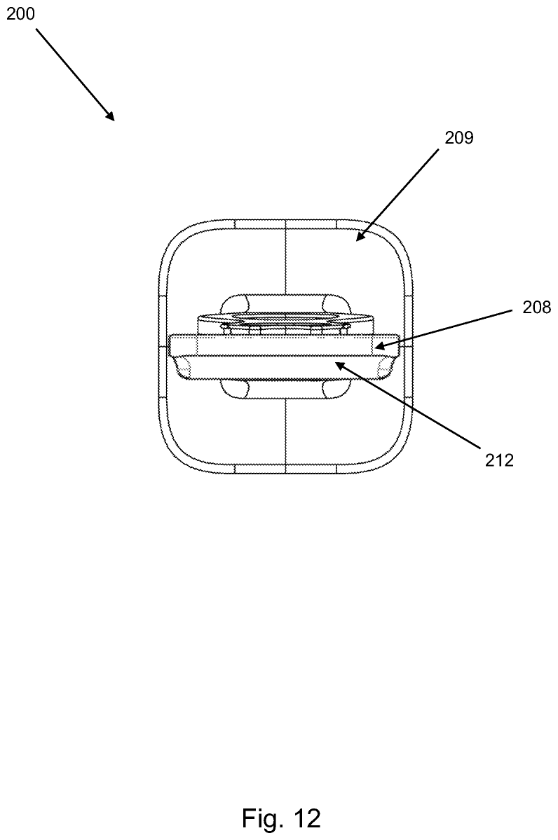

FIG. 12 shows a top view of the second embodiment of the insertion aid apparatus 200. In this view, the diaper insert 208 is visible with the pocket 212 installed over body 202. Base 209 provides stable and frictional support for apparatus 200.

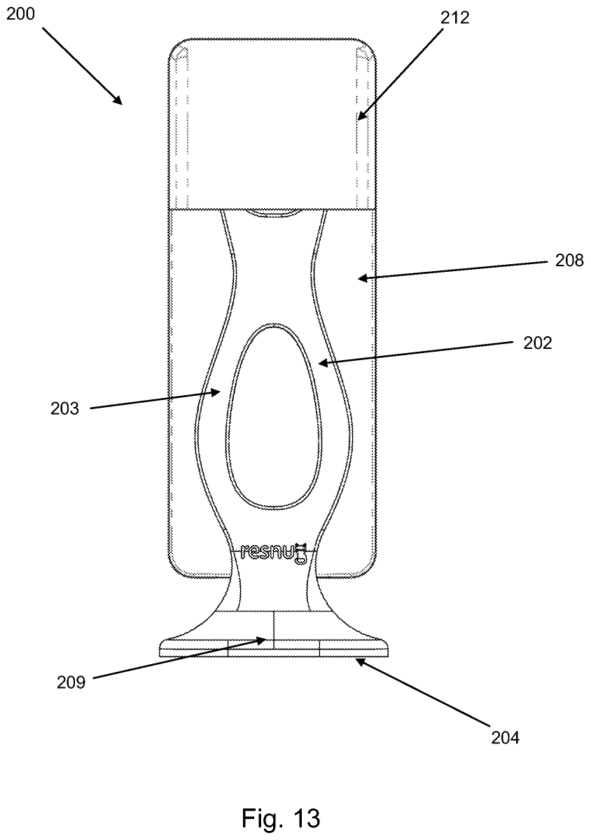

FIG. 13 shows a rear view of the second embodiment of the insertion aid apparatus 200. In this view, the diaper insert 208 is visible with the pocket 212 installed over body 202 as supported by vertical support 203. Base 209 provides stable and frictional support for apparatus 200 by utilizing pads 204.

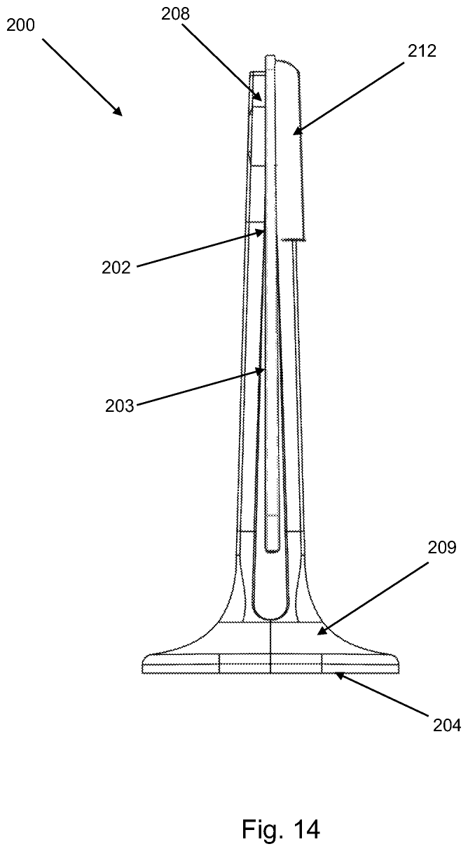

FIG. 14 shows a side view of the second embodiment of the insertion aid apparatus 200. In this view, the diaper insert 208 is visible with the pocket 212 installed over body 202 as supported by vertical support 203. Base 209 provides stable and frictional support for apparatus 200 by utilizing pads 204.

The present invention offers several key advantages over traditional methods of inserting cloth diaper inserts. Firstly, it significantly reduces the manual effort and time required for diaper preparation, making it more convenient and less physically demanding, particularly for individuals with limited hand dexterity or those dealing with multiple diapers.

Secondly, the apparatus's design allows for the insertion of multiple inserts simultaneously, further enhancing efficiency. Thirdly, the incorporation of a pocket in the insert design and the handle on the apparatus provides better grip and control during insertion, ensuring a secure and accurate placement.

Finally, the versatile design of the invention allows it to accommodate various insert configurations and potentially find applications in other textile manufacturing processes, demonstrating its broader utility beyond cloth diapering. Overall, the present invention represents a significant advancement in the field of cloth diapering, promoting ease of use, efficiency, and accessibility.

Additionally, the present insertion aid apparatus may be utilized to place other replaceable inserts into a corresponding fabric or deformable pocket of items. Such uses include: 1) inserting removable bra pads into sports bras, 2) inserting pads into athletic gear, 3) inserting pads into corresponding pockets in reusable feminine hygiene products, 4) inserting pads/padding into swimwear, 5) inserting pads or prosthetic inserts into mastectomy bras or post-surgery compression garments, 6) inserting padding into protective sports apparel such as cycling shorts, motorcycle jackets, or other protective gear, 7) inserting pads into adult incontinence garments, 8) inserting comforters into duvet covers, and 9) inserting pillows into pillow covers. While suitable for the above uses, the insert aid apparatus may be utilized for placing any removable or replaceable insert into any corresponding fabric or deformable pocket on any suitable item.

Although the present invention has been described in relation to the above disclosed preferred embodiments, many modifications in design, implementation, systems and execution are possible while still maintaining the novel features and advantages of the invention. The preferred embodiment is not meant to limit the scope of the patent in any way, and it should be given the broadest possible interpretation consistent with the language of the disclosure on the whole.

1. An apparatus for inserting a replaceable insert into a corresponding pocket comprising:
a base having a top and a bottom;
a body comprised of a first vertical support and a second vertical support,
wherein the body forms a slot for the insert formed by the first vertical support,
the second vertical support and the top of the base; and
and the base further comprising one or more pads installed on the bottom of the base.

2. The apparatus of claim 1 further comprising a fastening mechanism.

3. The apparatus of claim 2 where the fastening mechanism is selected from the group consisting of hook and loop material, clips, snaps, buttons, and magnets.

4. The apparatus of claim 1 where the pads are comprised of a slip-resistant material.

5. The apparatus of claim 4 where the slip-resistant material is selected from the group consisting of silicone, rubber, plastic, PVC, elastomer and felt.

6. The apparatus of claim 1 where the replaceable insert is selected from the group consisting of removable pads for sports bras, pads for athletic apparel, pads for reusable feminine hygiene products, pads for swimwear, pads for mastectomy bras, pads for post-surgery compression garments, pads for adult incontinence garments, comforters for duvet covers, and pillows for pillow covers.

7. The apparatus of claim 1 where the apparatus is made of a material selected from the group consisting of plastic, wood, and metal.

8. The apparatus of claim 1 where the apparatus is manufactured using a method selected from the group consisting of molding, cutting, 3d printing, and casting.

9. An apparatus for inserting a replaceable insert into a corresponding deformable pocket comprising:
a base having a top and a bottom;
a body comprised of a first vertical support and a second vertical support,
wherein the body forms a slot for the insert formed by the first vertical support,
the second vertical support and the top of the base; and
and the base further comprising one or more pads installed on the bottom of the base.

10. The apparatus of claim 9 further comprising a fastening mechanism.

11. The apparatus of claim 10 where the fastening mechanism is selected from the group consisting of hook and loop material, clips, snaps, buttons, and magnets.

12. The apparatus of claim 9 where the pads are comprised of a slip-resistant material.

13. The apparatus of claim 9 where the slip-resistant material is selected from the group consisting of silicone, rubber, plastic, PVC, elastomer and felt.

14. The apparatus of claim 9 where the replaceable insert is selected from the group consisting of removable pads for sports bras, pads for athletic apparel, pads for reusable feminine hygiene products, pads for swimwear, pads for mastectomy bras, pads for post-surgery compression garments, pads for adult incontinence garments, comforters for duvet covers, and pillows for pillow covers.

15. The apparatus of claim 9 where the apparatus is made of a material selected from the group consisting of plastic, wood, and metal.

16. The apparatus of claim 9 where the apparatus is manufactured using a method selected from the group consisting of molding, cutting, 3d printing, and casting.
